(19) United States
(12) Patent Application Publication
(10) Pub. No.: US 2022/0168463 A1
(43) Pub. Date: Jun. 2, 2022

(54) ENHANCED PERFORMANCE SOLUTION FOR TISSUE GRAFTS

(71) Applicant: The Curators of the University of Missouri, Columbia, MO (US)

(72) Inventors: Sheila Grant, Columbia, MO (US); David Grant, Columbia, MO (US); Colten Snider, Columbia, MO (US); Mitchell Bellrichard, Jonesburg, MO (US); Daniel Grant, Columbia, MO (US)

(21) Appl. No.: 17/538,733

(22) Filed: Nov. 30, 2021

Related U.S. Application Data
(60) Provisional application No. 63/119,197, filed on Nov. 30, 2020.

(51) Int. Cl. A61L 27/04; A61F 2/24; A61F 2/04; A61F 2/10

(52) U.S. Cl. CPC A61L 27/04; A61F 2/24; B82Y 5/00; A61F 2/105; A61F 2/042

(57) ABSTRACT

The present disclosure describes a treatment composition comprising a nanoparticle composition comprising nanoparticles functionalized with surface amine groups and a crosslinking composition comprising genipin. The disclosure also describes a kit comprising the treatment composition, and instructions for using the kit to crosslink the nanoparticles to a tissue graft. The treatment composition and kit can be used to crosslink nanoparticles to a tissue graft, and the resulting tissue graft can be used to replace defective tissue in a subject in need thereof.

### REFERENCE TO RELATED APPLICATIONS

This application claims the benefit of U.S. Provisional Patent Application No. 63/119,197, filed Nov. 30, 2020, the entire disclosure of which is incorporated herein by reference.

### STATEMENT REGARDING FEDERALLY SPONSORED RESEARCH OR DEVELOPMENT

None.

### FIELD OF INVENTION

The present disclosure relates solutions and kits and their use to crosslink nanoparticles to a tissue graft. More specifically, the present disclosure relates to their use in a surgical suit to crosslink nanoparticles to a tissue graft to be used to replace defective tissue in a subject in need thereof.

### BACKGROUND OF INVENTION

Tissue engineering has advanced as a promising solution for the repair of damaged or diseased tissues with the goal of creating functional scaffolds that mimic native tissue and can be colonized by the host's cells. Tissue grafts such as decellularized tissue have shown promise in this regard, and there are numerous surgical scaffolds in clinic use today utilizing both allogenic and xenogeneic decellularized tissue including, urinary bladder, small intestine, dermis, mesothelium, heart valves, and pericardium. An end goal is the use of three-dimensional scaffolds created through whole organ decellularization as a treatment for end-stage organ failure without the risk of chronic rejection and the morbidity associated with immunosuppression. Biological tissue is better able to mimic the full complexity of the tissue architecture while also being a source of signaling molecules and growth factors creating a superb environment for cellular attachment and proliferation. Although decellularized tissue has numerous promising characteristics, it is not without its drawbacks. These concerns include decellularization weakening mechanical properties, inherent heterogeneity, high immunogenicity, rapid biodegradation, and slow integration. This is specifically true with ligament grafts. Decellularized ligament and tendon use is limited due to a prolonged inflammatory period and delayed graft remodeling. A potential solution to some of these concerns is the utilization of nanoparticles, specifically gold nanoparticles.

Gold nanoparticles (AuNPs), conjugated to decellularized tissue, can mitigate some of the concerns with decellularized tissue grafts. First, AuNPs have long been utilized for their anti-inflammatory properties, which is believed to be the result of free radical scavenging. Secondly, gold nanoparticle attachment modifies the surface structure and encourage cellular attachment and proliferation. The increased surface energy of AuNPs may promote the attachment of proteins including those necessary for cellular attachment. In addition, conjugation of the AuNPs to the tissue is believed to block collagenase attachment and thereby slow down scaffold degradation. AuNPs can also be used to direct the differentiation of stem cells into specific cell types. AuNPs promote the osteogenic differentiation of mesenchymal stem cells by activating the p38 mitogen-activated protein kinase pathway, and the addition of AuNPs promotes osteogenic differentiation of adipose-derived stem cells and results in significantly higher new bone formation in a rabbit model.

With all the promise of nanoparticles, their attachment to tissue remains challenging. The commonly used methods have cytotoxic byproducts and require multiple washes to remove them. In addition, the current conjugation protocols only enable a rather rough estimate of the amount of attached gold as many of the agents only react for a limited period of time. There is a need to find a biocompatible, stable, crosslinking agent to facilitate the attachment of nanoparticles.

### SUMMARY OF INVENTION

The disclosure provides a composition comprising a nanoparticle composition comprising nanoparticles functionalized with surface amine groups and a crosslinking composition comprising genipin.

In one aspect, a kit for crosslinking nanoparticles to a tissue graft, the kit comprising a nanoparticle composition comprising nanoparticles, a functionalization component comprising a ligand having a thiol group and an amine group, a crosslinking composition comprising genipin, a buffer solution, and instructions for using the kit to crosslink the nanoparticles to the tissue graft, wherein the instructions comprise instructions to contact the nanoparticle composition with the functionalization component to form a functionalized nanoparticle composition, and combine the crosslinking composition, the functionalized nanoparticle composition, and the buffer solution to form a incubating composition and contact the tissue graft with the incubating composition is described.

In another aspect, a method of crosslinking nanoparticles to a tissue graft is described. The method comprises: preparing a treatment composition by combining a nanoparticle composition comprising nanoparticles functionalized with surface amine groups, a crosslinking composition comprising genipin, and a buffer solution comprising phosphate buffered saline; incubating the tissue graft in the treatment composition for at least 15 minutes; and rinsing the tissue graft, preferably, with sterile saline.

In yet another aspect, a method of replacing defective tissue in a subject in need thereof is described. The method comprises the method of crosslinking nanoparticles to a tissue graft and further comprising surgically implanting the rinsed nanoparticle-crosslinked tissue graft into the subject in need thereof in proximity to the defective tissue.

### BRIEF DESCRIPTION OF DRAWINGS

FIG. 1 depicts results of modulated differential scanning calorimetry in which the mean denaturation temperature of each tissue is shown. * indicates significantly different from all other treatment types. #indicates significantly greater than the untreated scaffold (p<0.05). Error bar displays standard error of the mean.

FIG. 2 depicts cell proliferation reagent WST-1 assay results showing percent viability relative to the control, untreated tissue scaffolds. * indicates significantly different from untreated control (p<0.05). Error bar displays standard error of the mean.

FIG. 3 depicts DNA content on scaffolds plated with L929 mouse fibroblasts for 1 to 10 days. (α) indicates significantly higher DNA content than 15 minutes or 1 hour crosslinking samples. (β) indicates significantly higher DNA content than the untreated samples. (p<0.05). Error bar displays standard error of the mean.

FIG. 4A is a scanning electron microscopy image of fibroblasts plated for 1 day on an uncrosslinked scaffold.

FIG. 4B is a scanning electron microscopy image of fibroblasts plated for 1 day on a scaffold made from 24 hour crosslinking with 3 mM genipin and 20 nm gold nanoparticles.

FIG. 4C is a scanning electron microscopy image of fibroblasts plated for 1 day on a scaffold made from 24 hour crosslinking with 3 mM genipin and without nanoparticles.

FIG. 4D is a scanning electron microscopy image of fibroblasts plated for 3 days on an uncrosslinked scaffold.

FIG. 4E is a scanning electron microscopy image of fibroblasts plated for 3 days on a scaffold made from 24 hour crosslinking with 3 mM genipin and 20 nm gold nanoparticles.

FIG. 4F is a scanning electron microscopy image of fibroblasts plated for 3 days on a scaffold made from 24 hour crosslinking with 3 mM genipin and without nanoparticles.

FIG. 4G is a scanning electron microscopy image of fibroblasts plated for 3 days on an uncrosslinked scaffold at increased magnification compared to FIG. 4D.

FIG. 4H is a scanning electron microscopy image of fibroblasts plated for 3 days on a scaffold made from 24 hour crosslinking with 3 mM genipin and 20 nm gold nanoparticles at increased magnification compared to FIG. 4E.

FIG. 4I is a scanning electron microscopy image of fibroblasts plated for 3 days on a scaffold made from 24 hour crosslinking with 3 mM genipin and without nanoparticles at increased magnification compared to FIG. 4F.

FIG. 5 is scanning electron microscopy images of gold nanoparticles conjugated with genipin to decellularized diaphragm tendon with crosslinking for 15 minutes.

### DETAILED DESCRIPTION OF INVENTION

This disclosure is directed to a composition comprising a nanoparticle composition and a crosslinking composition comprising genipin. This composition can be included in an intraoperative kit that can modify biological grafts in the surgical suite setting. The intraoperative kit allows conjugation of nanoparticles, particularly gold nanoparticles, to the grafts in less than 15 minutes followed by rinsing (e.g., in sterile saline solution) prior to implantation. The biofabrication method of conjugating nanoparticles to the tissue grafts within minutes in a surgical suite without the need for long-term extensive washing creates a novel modified biologic graft. The conjugation of nanoparticles to the graft will promote graft assimilation by reducing inflammation and encouraging cellular attachment and proliferation.

Tissue grafts such as decellularized allograft tissues are used for a wide array of tissue injuries and repair with tendons and ligament repair being among the most common. However, despite their frequent use there is concern over the lengthy inflammatory period and slow healing associated with allografts. One promising solution has been the use of nanoparticles. There is currently no easy, fast method to achieve consistent conjugation of nanoparticles to tissue. The available conjugation methods can be time-consuming and/or may create numerous cytotoxic byproducts. Genipin, a naturally derived crosslinking agent isolated from the fruits of Gardenia jasminoides was investigated as a conjugation agent to achieve fast, consistent crosslinking without cytotoxic byproducts. It is a natural crosslinking agent, and it spontaneously reacts with amino-group-containing compounds such as proteins, collagens, and gelatins to form mono-crosslinks to tetramer crosslinks, and has an exceptionally low cytotoxicity. Genipin also has the added benefit of acting as an anti-inflammatory agent and simultaneously reducing the immunogenicity of the scaffold. This disclosure shows that genipin is a viable agent to conjugate gold nanoparticles to tissue grafts quickly and efficiently.

### Compositions

One aspect of the disclosure relates to a treatment composition comprising a nanoparticle composition comprising nanoparticles functionalized with surface amine groups and a crosslinking composition comprising genipin.

This treatment composition can further comprise a biocompatible solvent. For example, phosphate buffered saline (PBS), N-(2-hydroxyethyl)piperazine-N′-(2-ethanesulfonic acid) (HEPES), 4-morpholinepropanesulfonic acid (MOPS), 2-(N-morpholino)ethansulfonic acid (MES), Dulbecco's phosphate buffered saline (DPBS), or combinations thereof can be added to the treatment composition. Phosphate buffered saline (PBS) is the preferred buffer and is a buffer solution commonly used in biological research. PBS can be prepared from a tablet to produce a solution of 10 mM phosphate, 2.7 mM KCl, and 137 mM NaCl with a pH of 7.5. Another exemplary formula of 1× PBS is 137 mM NaCl, 2.7 mM KCl, 10 mM Na₂HPO₄, and 1.8 mM KH₂PO₄. This PBS can also be supplemented with 1 mM CaCl₂·2H₂O and 0.5 mM MgCl₂·6H₂O. Its pH is adjusted to 7.4.

The treatment composition can further comprise at least one of an antimicrobial agent, an anti-inflammatory agent, a cell culture medium, or a combination thereof.

A preferred antimicrobial agent is EDTA. Other antimicrobial agents that can be used include antibiotics such as gentamicin or amphotericin, other agents commonly added to cell culture media such as normocin and streptomycin, and bacterial cell wall inhibitors.

The anti-inflammatory agent can comprise epigallocatechin-gallate (EGCG), curcumin, onion extract, pycnogenol, willow bark extract, Boswellia serrata resin, resveratrol, Uncaria tomentosa extract, capsaicin, or a combination thereof. The anti-inflammatory agent can be a natural compound such as a plant, herb, or plant or herb extract that works by inhibiting the inflammatory pathways in a similar manner as NSAIDs such as inhibiting the nuclear factor-kB inflammatory pathways.

The nanoparticles functionalized with surface amine groups can be any nanoparticles functionalized with surface amine groups as described herein. The nanoparticles are preferably functionalized with surface amino groups via addition of 2-mercaptoethylamine (i.e., cysteamine); however, they can be functionalized with any appropriate functionalization method as described herein.

The nanoparticles are preferably gold nanoparticles having a mean diameter of about 20 nm and having a spherical shape.

The nanoparticle composition can further comprise a biocompatible solvent. The biocompatible solvent can be water, PBS, saline solution, cell culture medium, or a combination thereof. The biocompatible solvent

Preferably, the nanoparticles in the nanoparticle composition have a concentration of 7.0×10¹¹ nanoparticles/mL (1×). The nanoparticles can range from 1.75×10¹¹ nanoparticles/mL (0.25×) to 5.6×10¹² nanoparticles/mL (8×).

### Kits

Another aspect of the disclosure relates to a kit for crosslinking nanoparticles to a tissue graft. The kit can comprise a nanoparticle composition comprising nanoparticles, a functionalization component comprising a ligand having a thiol group and an amine group, a crosslinking composition comprising genipin, a buffer solution, and instructions for using the kit to crosslink the nanoparticles to the tissue graft, wherein the instructions comprise instructions to contact the nanoparticle composition with the functionalization component to form a functionalized nanoparticle composition, and combine the crosslinking composition, the functionalized nanoparticle composition, and the buffer solution to form a incubating composition and contact the tissue graft with the incubating composition.

Further, another aspect includes a kit comprising a nanoparticle composition comprising nanoparticles functionalized with surface amine groups, a crosslinking composition comprising genipin, and a buffer solution, and instructions for using the kit to crosslink the nanoparticles to the tissue graft, wherein the instructions comprise instructions to incubate the tissue graft in a combination of the nanoparticle composition, the crosslinking composition, and the buffer solution.

The nanoparticles functionalized with surface amine groups, genipin, and buffer solution can be the same as those described herein and have the same properties.

For example, the combination of the functionalized nanoparticle composition, the crosslinking composition, and the buffer solution can have a nanoparticle concentration of 7.0×10¹¹ nanoparticles/mL (1×). The nanoparticles can range from 1.75×10¹¹ nanoparticles/mL (0.25×) to 5.6×10¹² nanoparticles/mL (8×). The concentration of nanoparticles in the nanoparticle composition comprising nanoparticles can be any concentration higher than the desired concentration in the combination of the three solutions such that it achieves the desired concentration when diluted with the other solutions.

Similarly, the combination of the functionalized nanoparticle composition, the crosslinking composition, and the buffer solution can have a genipin concentration from about 0.01 mM to about 10 mM; from about 0.01 mM to about 8 mM; from about 0.01 mM to about 6 mM; from about 0.01 mM to about 5 mM; from about 0.01 mM to about 4 mM; from about 1 mM to about 10 mM; from about 1 mM to about 8 mM; from about 1 mM to about 6 mM; from about 1 mM to about 5 mM; from about 1 mM to about 4 mM; or from about 1 mM to about 3 mM. Preferably, the combination has a genipin concentration of 3 mM. When the crosslinking composition is provided as a solution, the concentration of genipin in the crosslinking composition comprising genipin can be any concentration higher than the desired concentration in the combination of the nanoparticle composition, the crosslinking composition, and the buffer solution such that it achieves the desired concentration when diluted with the other solutions.

The nanoparticle composition can further comprise a biocompatible solvent or other carrier. For example, the biocompatible solvent can be water, phosphate buffered saline, cell culture medium, or a combination thereof. The biocompatible solvent can have a pH range of 5 to 9.

The functionalization component can be provided as a dry powder. The ligand having a thiol group and an amine group comprises methionine, mercaptoalkylamines such as mercaptomethylamine, 2-mercaptoethylamine (MEA), i.e., cysteamine, mercaptopropylamine, mercaptobutylamine, or a combination thereof; the ligand preferably comprises 2-mercaptoethylamine.

The crosslinking composition can be provided as a dry powder. The crosslinking composition can also further comprise a biocompatible solvent. The biocompatible solvent can be dimethyl sulfoxide (DMSO), ethanol, dimethyl formamine, methanol, or acetone. The biocompatible solvent is preferably dimethyl sulfoxide. The crosslinking composition can also further comprise a biocompatible carrier comprising water, phosphate buffered saline, cell culture medium, or a combination thereof.

The instructions can further comprise instructions to use the kit in a surgical suite as close temporally as possible to a procedure for implanting the tissue graft.

### Genipin

Genipin is a naturally derived crosslinking agent isolated from the fruits of Gardenia jasminoides that can be used for fast, consistent crosslinking without cytotoxic byproducts. It spontaneously reacts with amino-group-containing compounds such as proteins, collagens, and gelatins to form monomer to tetramer crosslinks, and has an exceptionally low cytotoxicity. Genipin can also act as an anti-inflammatory agent and can reduce the immunogenicity of the scaffold. Genipin is also known as methyl (1S,2R,6S)-2-hydroxy-9-(hydroxymethyl)-3-oxabicyclo[4.3.0]nona-4,8-diene-5-carboxylate and has the following chemical structure:

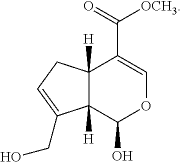 [SMILES: COC(=O)C1=CO[C@@H](O)[C@@H]2C(CO)=CC[C@H]12]

Genipin can be used in crosslinking applications such as those described herein. Because genipin crosslinking does not produce cytotoxic byproducts, solutions and methods utilizing it can be nontoxic and/or biocompatible.

### Nanoparticles

### Nanoparticle Properties

The nanoparticles described herein may be selected from a variety of nanoparticles, such as metallic nanoparticles, ceramic nanoparticles, polymer nanoparticles, and combinations thereof.

The metallic nanoparticles can comprise a material selected from the group consisting of gold, zinc oxide, silver, titanium, copper, selenium, nickel, platinum, zinc peroxide, magnesium oxide, cerium oxide, titanium dioxide, and combinations thereof.

The ceramic nanoparticles can comprise at least one material selected from the group consisting of oxides, carbides, phosphates and carbonates of metals and metalloids such as calcium, titanium, and silicon. The ceramic nanoparticles can comprise magnesium oxide, cerium oxide, graphene, carbon nanotubes, or combinations thereof.

The polymer nanoparticles can comprise at least one material selected from the group consisting of a degradable polymer and an anionic copolymer. Examples of suitable degradable polymer nanoparticles include nanoparticles comprising at least one of polycaprolactone, polylactic acid, polyglycolic acid, and polylactic glyocolic acid. Examples of suitable anionic copolymer nanoparticles include nanoparticles comprising at least one of methacrylic acid, polyethene glycol, poly(propylene glycol) (PPG), poly(lactic-co-glycolic acid) (PLGA)-(polyethylene glycol) PEG copolymer, or a derivative thereof.

The nanoparticles described herein may be selected from a variety of nanoparticles that are nontoxic and biocompatible such as gold, silver, silicon carbide, silicon, silica, and combinations of coated nanoparticles.

Other examples of suitable nanoparticles include silicon nanoparticles, silica nanoparticles, alumina nanoparticles, calcium phosphate nanoparticles, BaTiO₃ nanoparticles, or combinations thereof.

Preferably, the nanoparticles are gold nanoparticles.

The nanoparticles can be shaped as spheres, cages, rods, stars, clusters, tubes, polygons, pyramids, rings, or combinations thereof. Preferably, the nanoparticles are spheres.

The nanoparticles can have a mean diameter from about 5 nm to about 100 nm; from about 5 nm to about 90 nm; from about 5 nm to about 80 nm; from about 5 nm to about 70 nm; from about 5 nm to about 60 nm; from about 5 nm to about 50 nm; from about 5 nm to about 40 nm; from about 5 nm to about 30 nm; from about 10 nm to about 100 nm; from about 10 nm to about 90 nm; from about 10 nm to about 80 nm; from about 10 nm to about 70 nm; from about 10 nm to about 60 nm; from about 10 nm to about 50 nm; from about 10 nm to about 40 nm; from about 10 nm to about 30 nm; from about 15 nm to about 30 nm; from about 15 nm to about 25 nm; or about 20 nm. The nanoparticles preferably have a mean diameter of about 20 nm.

### Nanoparticle Functionalization

In the functionalizing step, the selected nanoparticles obtained commercially or synthesized according to various procedures in the field can be exposed to a plasma environment with selected plasma chemistry in order to introduce new functionalities which will enhance the bonding between the nanoparticles and tissue. Generally, the precursor selected for plasma polymerization is a molecule that has one or more of the desired functional groups and one or more carbon-carbon double bonds.

For example, when the desired surface functional group is an amine, the precursor would contain an amine and a carbon-carbon double bond. Examples of amines that can be used in plasma polymerization are allylamine, poly(allylamine), diaminocyclohexane, 1,3-diaminopropane, heptylamine, ethylenediamine, butylamine, propargylamine, propylamine, and the like. In some embodiments, amines that can be used in plasma polymerization are poly(allylamine), diaminocyclohexane, 1,3-diaminopropane, heptylamine, ethylenediamine, butylamine, propargylamine, propylamine, and the like.

Further, the functional groups, such as —NHx (x=1 or 2), are selected to act as anchoring points for crosslinking tissue grafts via covalent bond formation. A variety of plasma chemistry may be employed to introduce the functional groups. For example, allylamine may be used to deposit —NH, and, —NH₂ containing plasma coatings on the nanoparticle surfaces. Additionally, organosilicons including trimethylsilane (3MS) and hexa-methyldisiloxane (HMDSO) may be used to plasma coat the nanoparticles to ensure excellent adhesion of plasma coating to nanoparticles. The organosilicon coating provides a layer on the nanoparticle that aids adhesion of the nanoparticle to the deposited functionalized coating. Subsequent plasma treatment using O₂ or CO₂ may be used to further increase the surface concentration of these functional groups.

For silver nanoparticles or the preferred gold nanoparticles, the nanoparticles can be functionalized by coordinating a ligand containing the desired functional group to the gold or silver atom. Generally, the ligand should have at least two functional groups; one of the functional groups can coordinate to the metal site and the other could be used to crosslink with a tissue graft. For example, a ligand having a thiol group and an amine group; e.g., methionine, mercaptoalkylamines such as mercaptomethylamine, 2-mercaptoethylamine (MEA), i.e., cysteamine, mercaptopropylamine, mercaptobutylamine, and the like, can be coordinated to the metal of the nanoparticle to provide a functional group for further reaction with a tissue graft. Also, a ligand having a thiol group and a carboxylic acid group; e.g., thiosalicylic acid, 2-mercaptobenzoic acid, can be coordinated to the metal of the nanoparticle to provide a functional group for further reaction with a tissue graft.

The functionalizing of gold nanoparticles preferably occurs via addition of 2-mercaptoethylamine (also known as cysteamine). The 2-mercaptoethylamine can be added to the gold nanoparticles in water at a concentration of from about 0.0005 mg/mL to about 0.01 mg/mL; preferably, about 0.001 mg/mL 2-mercaptoethylamine.

When the nanoparticle is silicon carbide, the silicon carbide nanoparticle can be treated with various reagents that have at least two functional groups; one group that can react with the surface hydroxy groups on the silicon carbide and another functional group that can crosslink to a tissue graft. For example, the silicon carbide particles can be reacted with aminoalkyl-trialkoxysilanes such as aminomethyl-trimethoxysilane, aminoethyl-trimethoxysilane, aminopropyl-trimethoxysilane, aminobutyl-trimethoxysilane, aminomethyl-triethoxysilane, aminoethyl-triethoxysilane, aminopropyl-triethoxysilane, aminobutyl-triethoxysilane, aminomethyl-tripropoxysilane, aminoethyl-tripropoxysilane, aminopropyl-tripropoxysilane, aminobutyl-tripropoxysilane, aminomethyl-tributoxysilane, aminoethyl-tributoxysilane, aminopropyl-tributoxysilane, aminobutyl-tributoxysilane, or a combination thereof to provide amine groups on the surface of the silicon carbide nanoparticle.

The functionalization of the gold nanoparticles produces nanoparticles that have from about 1×10⁻¹⁰ mol/cm² to about 1×10⁻⁹ mol/cm²; from about 2×10⁻¹⁰ mol/cm² to about 1×10⁻⁹ mol/cm² or from about 5×10⁻¹⁰ mol/cm² to about 1×10⁻⁹ mol/cm² functional groups per gold nanoparticle.

### Tissue Grafts

A tissue graft can be obtained commercially (i.e. tissue bank, etc.), generated via a 3D printing apparatus, or harvested via allografts, xenografts, or from the subject who is the intended recipient of the tissue graft. An allograft is harvested from an organism of the same species of the subject who is the intended recipient of the tissue graft. A xenograft is harvested from an organism of a different species of the subject who is the intended recipient of the tissue graft. An allograft or xenograft tissue graft is decellularized in that cells and cellular remnants are removed while the extracellular matrix components remain intact as described herein.

A variety of biological tissue donor sources may be employed, such as human (dermis, tensor fascia lata, blood vessels, and amniotic membrane), porcine (small intestine submucosa, dermis, blood vessels, and bladder), bovine (dermis, blood vessels, and pericardium), and equine (blood vessels and pericardium), which have been studied for other purposes. Many of these materials provide desirable degradation characteristics and when implanted either alone or once crosslinked to nanoparticles, can release growth factors and peptides that possess antimicrobial properties, enhance angiogenesis, and aid tissue remodeling by attracting endothelial and bone marrow-derived cells to the implant site.

The tissue graft can be a soft tissue. The soft tissue can comprise a muscle, a tendon, a ligament, an adipose tissue, a lymphatic vessel, a blood vessel, a fascia, a synovial membrane, or combinations thereof. For example, the tissue graft can be porcine diaphragm tendon tissue or an anterior cruciate ligament. The tissue graft can also comprise urinary bladder, small intestine, dermis, mesothelium, heart valve, or pericardium tissue.

The tissue may be selected according to its handling properties for surgical manipulation and mechanical properties (strength, elasticity, size, etc.) required for the defective tissue repair application, such as soft tissue repair. For example, the thickness of the tissue affects its handling properties and tissues having a thickness of from about 2.9 mm to about 6.2 mm is preferred. Also, the tensile strength of the tissue graft measured at yield ranges from about 50 MPa to about 150 MPa. For commercialization purposes, a user may also consider whether large quantities of the tissue can be easily obtained and processed.

The mechanical and chemical properties of the tissue graft desirably do not change significantly once implanted in an animal. For example, the viscoelasticity of the tissue graft does not change significantly as cells from the surrounding tissue infiltrate the tissue graft and it degrades. In order to have a composite that has a desired viscoelasticity, the tissue should have an appropriate degradation rate. Further, the viscoelasticity can be measured by the Young's modulus wherein a higher value means the tissue is stiffer and a lower value means the tissue is less stiff. Preferably, the viscoelasticity of the tissue graft is from about 500 MPa to about 2 GPa.

In addition to these considerations, the degradation rate of the tissue can also influence the selection of a particular tissue. When utilized for soft tissue repair, it is important that the selected natural tissue is degraded by the body at a rate that matches the healing rate of the defective area so that it can serve as an effective repair material without inciting a chronic inflammatory response.

The tissue grafts can have a range of geometries depending on the desired use. For example, the tissue graft can be cut to fit the particular site either before or after crosslinking to the nanoparticles. Thus, the tissue graft can be a range of dimensions and shapes. For example, the tissue graft can be a regular or an irregular shape, namely, a square, rectangle, trapezoid, parallelogram, triangle, circle, ellipsoid, cylinder, barbell, or any irregular shape that is appropriate to the use thereof. The selected tissue graft may also be stored in a buffered solution containing protease inhibitors and bacteriostatic agents at pH about 8 and 4° C. to prevent degradation of the tissue by lysosomal enzymes released by the biological cells before proceeding with the method.

When the tissue graft is implanted at a desired site in an animal, there is typically an underlying layer of muscle, then the tissue graft implant and an overlying layer of tissue. Thus, immediately after the placement of the implant until the time that the implant has been completely absorbed by the body, these three layers will be present. Over time, the overlying tissue will migrate and infiltrate the implant and the border between the implant and the tissue will be compromised.

The biodegradability of the implanted tissue graft is usually determined by removing the tissue graft and surrounding tissue from the animal and performing a visual inspection of the margins between the underlying muscle and the implant as well as the overlying tissue and the implant. At a certain time after placement, the margin between the tissue (muscle or other tissue) and the implant will not be visible. At this point the implant is considered to be completely biodegraded. Preferably, the time for complete degradation of the implant is substantially the same as the healing time for the tissue. For example, the time for degradation ranges from about 1 month to about 12 months; from about 1 month to about 9 months; from about 1 month to about 6 months; from about 2 months to about 6 months; or from about 3 months to about 6 months.

The tissue graft is biocompatible. The biocompatibility of the tissue graft can be measured using flow cytometry wherein cells incubated with the tissue graft did not show a significantly higher cell death rate as compared to the same cells under the same conditions but without contacting a tissue. A significantly higher cell death rate occurs when statistical significance (p<0.05) is measured. Microscopic analyses may be performed to verify that all fibroblasts and endothelial cells are successfully removed from the resulting tissue. Methyl green pyronin stain, which stains for DNA and RNA, may also be utilized to verify that remnants of DNA and RNA are effectively removed from the tissue during the extensive rinse sequence. Further histological analyses, such as Masson's Trichrome, Verhoeff-van Gieson, and Alcian Blue staining, may also be performed to verify that ECM components remain within the tissue graft.

The selected biological tissues of an allograft or xenograft need to be processed to remove native cells, i.e. “decellularized” in order to prevent an immune response when it is utilized as a soft tissue repair material (Gilbert et al. Decellularization of tissues and organs. Biomaterials 2006; 27:3675-3683). Successful decellularization is characterized by the removal of cellular nuclei and remnants with the retention of natural extracellular matrix components (collagen, elastin, growth factors, etc.) and overall tissue structure (collagen architecture) (Gilbert et al.). For example, from about 80% to 100%, from about 85% to about 100%, from about 90% to about 100%, or from about 95% to about 100% of the cellular nuclei and remnants are removed from the tissue. Further, the decellularized material can contain from about 0.1% to about 20%; from about 0.1% to about 15%; from about 0.1% to about 10%; from about 0.1% to about 5% of the original cellular material after decellularization. The ECM structure is ideal for cell attachment and infiltration. Thus, maintaining the ECM structure is desirable during the decellularization process.

Decellularized tissue may be obtained commercially, or harvested tissue may be decellularized as described herein. The decellularization process may be optimized for each species and type of tissue.

Generally, the decellularization process includes immersion of the desired tissue in an agent that can make the tissue acellular (i.e., the tissue contains no cells). The tissue can be immersed in the agent for about 6 hours to about 36 hours; from about 12 hours to about 30 hours; from about 18 hours to about 30 hours; or from about 20 hours to 28 hours. The tissue can be immersed in the agent more than once, with fresh agent added between immersions. The decellularization process can be performed at room temperature.

### Method of Crosslinking Nanoparticles to a Tissue Graft

The disclosure further provides a method of crosslinking nanoparticles to a tissue graft. The inventive method includes the steps of preparing a treatment composition by combining a nanoparticle composition comprising nanoparticles functionalized with surface amine groups, a crosslinking composition comprising genipin, and a buffer solution (preferably, comprising phosphate buffered saline); incubating the tissue graft in the treatment composition for at least 15 minutes; and rinsing the tissue graft. This method can utilize any of the previously described compositions and kits described herein.

In the method, a tissue graft is incubated in a treatment composition comprising nanoparticles functionalized with surface amine groups, genipin, and a buffer solution (e.g., phosphate buffered saline). The nanoparticles functionalized with surface amine groups can be any of the nanoparticles described herein that are functionalized with surface amine groups. The genipin functions as a crosslinking agent that reacts with amines to crosslink the nanoparticles functionalized with surface amine groups and the tissue graft. The treatment composition can have a genipin concentration from about 0.01 mM to about 10 mM and the range of concentrations of genipin in the crosslinking compositions described herein. The genipin concentration in the treatment composition is preferably 3 mM.

The volume of the treatment composition varies depending on the size of the tissue graft but should be sufficient to cover the tissue graft during incubation. For example, the volume of the treatment composition can be 100 mL, 200 mL, 300 mL, 500 mL, or 1000 mL.

The genipin crosslinking can occur in about 15 minutes to about 24 hours. Preferably, the crosslinking occurs in about 15 minutes. The tissue graft is then rinsed with a sterile solution, preferably, with sterile saline, water, phosphate buffered saline, cell culture medium, or a combination thereof before further use.

The method can be implemented in a laboratory or clinical setting. Preferably, the method is implemented within a surgical suite.

The treatment composition can further comprise at least one of an antimicrobial agent, an anti-inflammatory agent, a cell culture media, or a combination thereof. These agents are described herein above.

Various concentrations of nanoparticles as described herein may be utilized in the crosslinking solution to achieve optimal crosslinking. The incubation can occur at room temperature. The incubation can also occur on an orbital shaker table at low rpm. The method can further comprise storing the crosslinked tissue graft at 4° C. after rinsing with saline until the tissue graft can be utilized.

Generally, the sizes of the nanoparticles are selected to be substantially similar in size to the diameter of the fibers (e.g., collagen, elastin, fibronectin, laminin, glycosaminoglycans) in the tissue graft. When collagen fibers are present in the tissue graft, the collagen fibers have a diameter of about 30 nm. In particular, the nanoparticles have a mean diameter as described herein, and preferably have a mean diameter of about 20 nm.

Further, the nanoparticles can be distributed uniformly on the surface and/or within the tissue graft. Alternatively, the nanoparticles can be distributed nonuniformly on the surface and/or within the tissue graft.

Further, the particle sizes for the nanoparticles can be polydisperse or monodisperse. When gold nanoparticles are used, the nanoparticles can be monodisperse. Such a diameter for the nanoparticles provides a specific surface area of from about 8.6×10⁴ cm²/g to about 3.5×10⁵ cm²/g; from about 1×10⁵ cm²/g to about 2×10⁵ cm²/g or about 1.5×10⁵ cm²/g. These specific surface areas are for one nanoparticle, thus, the combined specific surface are of several nanoparticles in the tissue graft would be the specific surface area of one nanoparticle multiplied by the density of the nanoparticles in the tissue graft.

Further, the density of the nanoparticles on the surface of the tissue graft and/or within the tissue graft can be optimized to provide the appropriate surface area for cell growth, infiltration, and vascularization. When nanoparticles are used that have a mean diameter of from about 15 nm to about 30 nm, preferably 20 nm, the nanoparticles can infiltrate into the tissue graft and provide a surface for cell growth.

Further, for example, the nanoparticles crosslinked to the tissue graft can have a concentration of from about 15 μg/g to 25 μg/g, from about 50 μg/g to about 400 μg/g; from about 75 μg/g to about 400 μg/g; from about 100 μg/g to about 400 μg/g; from about 125 μg/g to about 400 μg/g; from about 150 μg/g to about 400 μg/g; from about 175 μg/g to about 400 μg/g; from about 200 μg/g to about 400 μg/g; from about 225 μg/g to about 400 μg/g; from about 250 μg/g to about 400 μg/g; from about 275 μg/g to about 400 μg/g; from about 300 μg/g to about 400 μg/g; from about 325 μg/g to about 400 μg/g; or from about 350 μg/g to about 400 μg/g.

The crosslinking density in the tissue graft can generally be measured by a collagenase assay wherein an increase in release of hydroxyproline indicates degradation of collagen. It would be expected that tissues that had lower crosslinking density would have a greater rate of collagen degradation and result in more hydroxyproline being released. Further, the mechanical properties can measure the crosslinking density wherein the tensile strength would be expected to increase with increasing crosslinking density. Further, the differential scanning calorimetry measurements indicate the crosslinking density of the material because a material that has a greater crosslinking density should have a higher denaturation temperature.

Depending on the chemical identity of the nanoparticles that are crosslinked to the tissue graft, the tissue graft can scavenge free radicals. For example, gold nanoparticles have the ability to scavenge free radicals. Without being bound by theory, it is believed that the free radical scavenging ability of the gold nanoparticles is able to ameliorate and/or reduce inflammation at the tissue graft implant site. The free radical scavenging capability of the gold nanoparticle tissue graft can be measured using the technique of Hsu et al., J. Biomedical Materials Research Part A 2006, 759. The capacity of the sample to scavenge can be measured by placing the sample (7.5 mm diameter, 1 mm thick) in 3 mL of 32 μM 2,2-diphenyl-1-picrylhydrazyl (DPPH), vortexed, and left to stand at room temperature for 90 minutes. The absorbance of the reaction mixture can be measured at 515 nm using a UV/VIS spectrophotometer and the following equation:

Scavenging ratio (%)=[1−Absorbance of test sample/Absorbance of control]×100%. Thus, the free radical scavenging ratio of the gold nanoparticle tissue graft is expected to be higher than the scavenging ratio of the tissue graft without gold nanoparticles.

Crosslinking of the nanoparticles to the tissue graft is joining the two components by a covalent bond. Crosslinking reagents are molecules that contain two or more reactive ends capable of chemically attaching to specific functional groups on proteins or other molecules (e.g., a tissue graft). These functional groups on the tissue graft are amines. To enhance the crosslinking between the selected nanoparticles and tissue graft, the functionalized nanoparticles with surface functional groups capable of bonding with tissue are preferred over the “naked” nanoparticles. Though a variety of functional groups may be selected, in particular, various functional groups that are capable of forming covalent peptide bonding with tissue, such as —NH, —NH₂, or a combination thereof, are employed.

Nanoparticles incorporated into the tissue graft improves the strength of the tissue graft and its resistance to degradation by the body, as well as influences cellular behavior and biocompatibility. Prior studies have demonstrated that nanoparticles are more hydrophilic and possess an increased number of atoms and crystal grains at their surface compared to conventional materials. The large number of grains at the surface leads to increased surface roughness, surface area, and surface energy which are thought to contribute to an increase in protein adsorption and unfolding. For example, nanoscale ceramics, metals, and polymers have all been shown to improve cellular function compared to conventional materials (Webster T J et al. J Biomed Mater Res 2000; 51:475-483; Price R L, et al. Journal of Biomedical Materials Research Part A 2003; 67A:1284-1293; Webster T J, et al. Biomaterials 2004; 25:4731-4739; Park G E, et al. Biomaterials 2005; 26:3075-3082; Thapa A, et al. Journal of Biomedical Materials Research Part A 2003; 67A:1374-1383; Christenson E M, et al. Journal of Orthopaedic Research 2007; 25:11-22). These properties make nanoparticles ideally suited to enhance the biocompatibility and cell/tissue interaction with tissue grafts.

The surface energy increase caused by the addition of nanoparticles is measured as compared to an otherwise identical tissue graft having micron-sized structures. Also, this surface energy increase is evidenced by increased protein adsorption as compared to an otherwise identical tissue graft having micron-sized structures. The identical tissue graft having micron-sized structures has the same matrix and chemical identity of the particles crosslinked to the matrix, but instead of nano-sized particles, the composite has micron-sized particles. The micron-sized material has a diameter of at least 100 nm. The protein adsorption can be measured by hematoxylin and eosin (H&E) stain of the composite followed by histology reading to quantify the amount of proteins adsorbed to the composition.

Optionally, in addition to the endogenous proteins, growth factors, and peptides that enhance cell adhesion, cell growth, and cell infiltration into the tissue graft, the functionalization of the nanoparticles may include a substep to increase tissue integration, wherein the nanoparticles may be treated with exogenous cell adhesion proteins and/or peptides. The addition of these active groups will promote better cellular adhesion, vascularization, and improve overall biocompatibility. The ECM proteins are important in cell adhesion. Cell adhesion to ECM proteins is mediated by integrins. Integrins bind to specific amino acid sequences on ECM proteins such as RGD (arginine, glycine, aspartic acid) motifs. Therefore, there has been research conducted on the control of the orientation and conformation of cell adhesion proteins onto materials so that RGD motifs are accessible to integrins. For example, fibronectin and fibronectin-III have been adsorbed onto synthetic surfaces. The results showed that presence of fibronectin-III displayed more cell-binding domains than the fibronectin-free surface. Thus, it is possible to manipulate and specifically orient the cell binding proteins so that increased tissue integration is possible. The ability of collagen type IV, fibronectin, and laminin type I to promote peri-implant angiogenesis and neovascularization has been studied; laminin stimulated extensive peri-implant angiogenesis and neovascularization into the porous ePTFE substrate material. Additionally, vascular endothelial growth factor (VEGF) is a chemical signal secreted by cells to stimulate neovascularization. VEGF stimulates the proliferation of endothelial cells. TGF-B1 (transforming growth factor) is another chemical signal that stimulates the differentiation of myofibroblasts. Both types of growth factors have been incorporated into tissue engineered scaffolds to stimulate and accelerate reconstitution of native tissue. The additional amines can be used as sites for attaching cell adhesion peptides, growth factors, glycosaminoglycans, or anti-inflammatory medications to further improve the biocompatibility of the nanoparticle-crosslinked tissue graft.

### Methods of Use

The inventive method of crosslinking a tissue graft may be used in a wide range of tissue engineering applications, including employing the tissue graft as a scaffold in tissue engineering and implanting the tissue draft in a living subject. Preferably, the method comprises replacing defective tissue in a subject in need thereof.

The method for replacing defective tissue in a subject in need thereof comprises: the previously described method of crosslinking nanoparticles to a tissue graft and further comprises surgically implanting the rinsed nanoparticle-crosslinked tissue graft into the subject in need thereof in proximity to the defective tissue. This method can utilize any of the previously described compositions, kits, or methods described herein.

The defective tissue can be a soft tissue. The soft tissue can comprise at least one muscle, tendon, ligament, adipose tissue, lymphatic vessel, blood vessel, fascia, synovial membrane, or combinations thereof. For example, the soft tissue can comprise diaphragm tendon tissue or anterior cruciate ligament. Additionally, the defective tissue can comprise urinary bladder, small intestine, dermis, mesothelium, heart valve, or pericardium tissue. The subject can also have a hernia to be repaired.

The subject can be a human, pig, cow, or horse. The subject is preferably a human. A decellularized tissue graft can originate from a species that is different from the species of the subject (i.e. xenograft) or from a species that is the same as the species of the subject (i.e. allograft). The tissue graft can also originate from the subject.

The replacing defective tissue in a subject in need thereof can further comprise infiltration of healthy cells from the subject into the tissue graft. The tissue graft can promote viability and/or proliferation of the cells as well as attachment of the cells to the tissue graft.

The method can further comprise delivering a healing agent selected from the group consisting of cells, growth factors, adhesion proteins, hormonal proteins, and combinations thereof from the tissue graft to the defective tissue. The healing agent can be delivered to the defective tissue by degradation of tissue graft or by desorption of the healing agent from the tissue graft.

Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety.

Having described the invention in detail, it will be apparent that modifications and variations are possible without departing from the scope of the invention defined in the appended claims.

### EXAMPLES

The following non-limiting examples are provided to further illustrate the present invention and further provides several examples of the solutions, kits, and methods of the present disclosure.

### Example 1: Tissue Harvest and Decellularization

The following materials and methods were used in performing the rest of the examples.

Porcine diaphragms were harvested immediately following euthanasia after a laboratory exercise at the University of Missouri. The central tendon portion of the diaphragm was dissected from the surrounding muscle. They were then decellularized according to a previously published protocol (Deeken, et al., J. Biomed. Mater. Res. Part B Appl. Biomater. 2011, 96, 351-359.). In brief, the tissues were immersed in a solution containing 1% (v/v) tri(n-butyl) phosphate (TnBP) (Sigma-Aldrich, St. Louis, Mo., USA) in storage buffer solution and subjected to continuous agitation on an orbital shaker at ambient temperature for 24 hours. The 1% TnBP solution was removed after 24 hours and exchanged with fresh solution, and the tissues were subjected to continuous agitation for an additional 24 hours. This treatment was followed by a 24 hour rinse with double distilled water and another 24 hour rinse with 70% (v/v) ethyl alcohol, both with continuous agitation at ambient temperature. This method of decellularization was previously verified to effectively remove all cell nuclei while leaving the structure and composition of the tissue intact. 4.8 mm circular discs were cut from the decellularized diaphragm tendon and stored in 70% (v/v) ethanol at 4° C.

### Example 2: Genipin Gold Nanoparticles and the Crosslinking Procedure

The following materials and methods were used in performing the rest of the examples.

Genipin (Sigma-Aldrich, St. Louis, Mo., USA) crosslinking was conducted by immersing the decellularized tissue into 1 mL of solution containing 3 mM or 10 mM dissolved genipin. The genipin was dissolved using dimethyl sulfoxide and suspended in PBS. This was accompanied with 0.25 mL of 20 nm gold nanoparticles (Ted Pella, Redding, Calif., USA) at a concentration of 7.0×10¹¹ nanoparticles/mL. The 20 nm AuNPs were utilized due to a previous study demonstrating the efficacy of nanoparticles in this size range in reducing inflammation. Nanoparticles were functionalized with amine groups by the addition of 0.001 mg/mL 2-mercaptoethylamine (Cysteamine) (Sigma-Aldrich, St. Louis, Mo., USA) to the nanoparticles. The scaffolds were crosslinked for 15 minutes, 1 hour, 4 hours or 24 hours and then were quickly rinsed with PBS. The AuNP tissue control samples were created using the same methodology with the exception that the genipin solution was replaced with PBS.

Tissue scaffolds were sterilized following crosslinking by immersion in 90% ethanol for 24 hours at 225 rpm. This was followed by three washes in sterilized phosphate buffered saline.

### Example 3: Experimental Groups

The following materials and methods were used in performing the rest of the examples.

Group 1 is untreated: porcine diaphragm tendon that underwent decellularization protocol.

Group 2 is 15 minutes, 1 hour, 4 hours, 8 hours, 24 hours Au Gen: decellularized tissue crosslinked with 0.25 mL of functionalized 20 nm gold nanoparticles at the stock and 1 mL of genipin at 3 mM. Crosslinking time ranged from 15 minutes to 24 hours.

Group 3 is 15 minutes, 1 hour, 4 hours, 8 hours, 24 hours Gen: decellularized tissue crosslinked with 0.25 mL of PBS and 1 mL of genipin at 3 mM. Crosslinking time ranged from 15 minutes to 24 hours. This group was crosslinked with genipin but without the addition of AuNPs.

Group 4 is 15 minutes, 1 hour, 4 hours, 8 hours, 24 hours Au: decellularized tissue crosslinked with 0.25 mL of functionalized 20 nm gold nanoparticles at the stock and 1 mL of PBS. Crosslinking time ranged from 15 minutes to 24 hours. This group was conjugated with nanoparticles but without the addition of genipin.

Group 5 is EDC/NHS crosslinked tissue: decellularized tissue that were crosslinked with the chemical crosslinkers 1-ethyl-3-[3-dimethylaminopropyl]carbodiimide (EDC) (Thermo Fisher Scientific, Waltham, Mass., USA) and N-hydroxysuccinimide (NHS) (Thermo Fisher Scientific, Waltham, Mass.) according to a previously published protocol (Nam, et al., Int. Immunopharmacol. 2010, 10, 493-499.). Briefly, 2 mM EDC 2-(N-Morpholino)ethanesulfonic acid buffer and combined with 5 mM NHS in dimethyl formamide. These were then added to a 50:50 (v/v) acetone:phosphate buffered saline mixture. The tissue was incubated in 0.25 mL of crosslinking solution for 15 minutes. The tissue was then incubated overnight and then rinsed for 48 hours in PBS at 225 rpm. EDC/NHS is a zero-length crosslinker commonly utilized to crosslinked tissue. It was utilized as a control in the DSC studies.

### Example 4: Cell Culture

The following materials and methods were used in performing the rest of the examples.

L-929 mouse fibroblast cells were obtained from ATCC (Manassas, Va.). They were cultured in EMEM (ATCC, Manassas, Va., USA) supplemented with 10% (v/v) horse serum (Sigma-Aldrich, St. Louis, Mo., USA) and 200 U mL⁻¹ penicillin-streptomycin (Sigma-Aldrich, St. Louis, Mo., USA) solution in an incubator at 37° C. and 5% CO2. 1 mL of 3×10⁴ cell/mL cell solution was plated on each scaffold and allowed to grow for 1, 3, 7, or 10 days with fresh media being replaced every 48 days.

### Example 5: Statistical Analysis

The following materials and methods were used in performing the rest of the examples.

GraphPad Prism 8.0.1 (GraphPad Software, San Diego, Calif., USA) was used to analyze experimental data. The Student's t test was used to analyze gold levels between samples with and without genipin. One-way analysis of variance was conducted followed by a Tukey-Kramer post-test to determine significant differences between means of the experimental groups. The results were considered statistically significant where p was less than 0.05.

### Example 6: Neutron Activation Analysis

NAA was utilized to quantify the gold levels in the tissue scaffolds. Following crosslinking, five samples of each treatment type (N=5) were lyophilized, weighed, and packed into high density polyethylene NAA vials. At the University of Missouri Research Reactor, the samples were loaded into a rabbit system with Au comparator standards and irradiated for 120 s in a thermal neutron flux of 5.0×1013 n/cm²/s. The ¹⁹⁷Au captures a neutron to produce the radio-isotope ¹⁹⁸Au with a 2.7 day half-life. The samples were allowed to decay for 1-7 hours and then counted for 10 minutes each using a high purity Ge detector controlled by Canberra Genie 2000 software. The detector dead-time was less than 5% for all samples.

NAA was performed to measure the concentration of gold attached to the tissue scaffold. As shown in Table 1 below, the number of nanoparticles increased with the amount of crosslinking time with or without the use of genipin. For all time points, the scaffold with genipin had a higher concentration of gold, but the difference was only significant at the 15 minute time point.

Table 1 shows Neutron Activation Analysis results. Concentration of gold (μg/g) on lyophilized tissue with and without the addition of genipin. * indicates constructs that have significantly higher concentration of AuNPs than the sample incubated for the same amount of time without genipin. Values are given as ±the standard deviation.

TABLE 1
Neutron Activation Analysis Results

| Conjugation Time | Au | Au and Genipin |
|---|---|---|
| 15 min | 51 | 100 * |
| 1 h | 86 | 92 |
| 4 h | 153 | 190 |
| 8 h | 224 | 228 |
| 24 h | 373 | 389 |

Example 7: Modulated Differential Scanning Calorimetry

Following crosslinking, five specimens (n=5) from the 7 treatment types were rinsed in DI water and placed in an aluminum pan with a hermetic lid. The treatment types were EDC/NHS, untreated, 15 minutes crosslinking with 3 mM genipin, 4 hours crosslinking with 3 mM genipin, 8 hours crosslinking with 3 mM genipin, 24 hours crosslinking with 3 mM genipin, and 24 hours crosslinking with 10 mM genipin. The 10 mM treatment type was added to verify that increasing the genipin concentration would further increase the amount of crosslinking that occurred. The reference pan consisted of an aluminum pan containing 2 μL of double distilled water and sealed with a hermetic lid. Each specimen was then subjected to modulated differential scanning calorimetry using a Q2000 DSC (TA Instruments, New Castle, Del., USA) to raise the temperature from 5° C. to 120° C. at a rate of 5° C. per minute with a modulation of ±0.64° C. every 80 s. The mean denaturation temperature is reported.

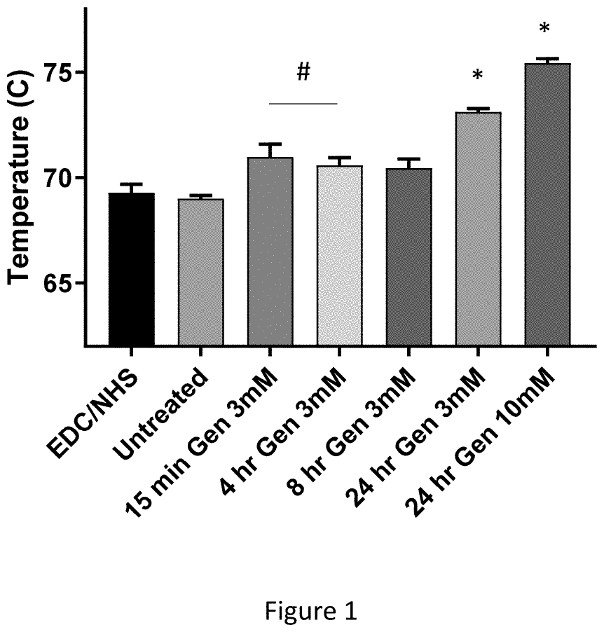

FIG. 1 displays the denaturation temperatures of the tissue scaffold with different crosslinking times and different concentrations of genipin. The denaturation temperature significantly increases with only 15 minutes of genipin crosslinking. Another significant increase was seen at 24 hours of crosslinking. Denaturation temperature also significantly increased with higher genipin concentrations.

### Example 8: Cell Viability

Cell proliferation reagent WST-1 (Roche Diagnostics Corporation, Indianapolis, Ind., USA) was used to evaluate the biocompatibility of the scaffolds. The WST-1 assay works via the use of tetrazolium salts. The salts were added to wells containing cells and the tissue discs. The tetrazolium salts were then cleaved to formazan by mitochondrial dehydrogenase activity. This correlates to the number of metabolically active cells. The resulting formazan was quantified using UV-vis absorbance measurements. A total of 5 scaffolds (N=5) from ten treatment types (untreated, 15 minutes, 1 hour, and 24 hours with 3 mM genipin and gold nanoparticles or either of the components independently) were seeded with L929 mouse connective tissue fibroblasts and incubated for 1, 3, 7, and 10 days with half of the media in each well replaced every 48 hours. WST-1 reagent was added to each well and the plates incubated at 37° C. for 4 hours. After gentle mixing, 100 μL was removed from each well and absorbance readings were acquired using a Tecan Safire II plate reader. The resulting values were then calculated relative to the absorbance found on the untreated control scaffold. The data is shown as a percentage in comparison to the control. Culture medium with the WST-1 reagent and no cells served as the blank.

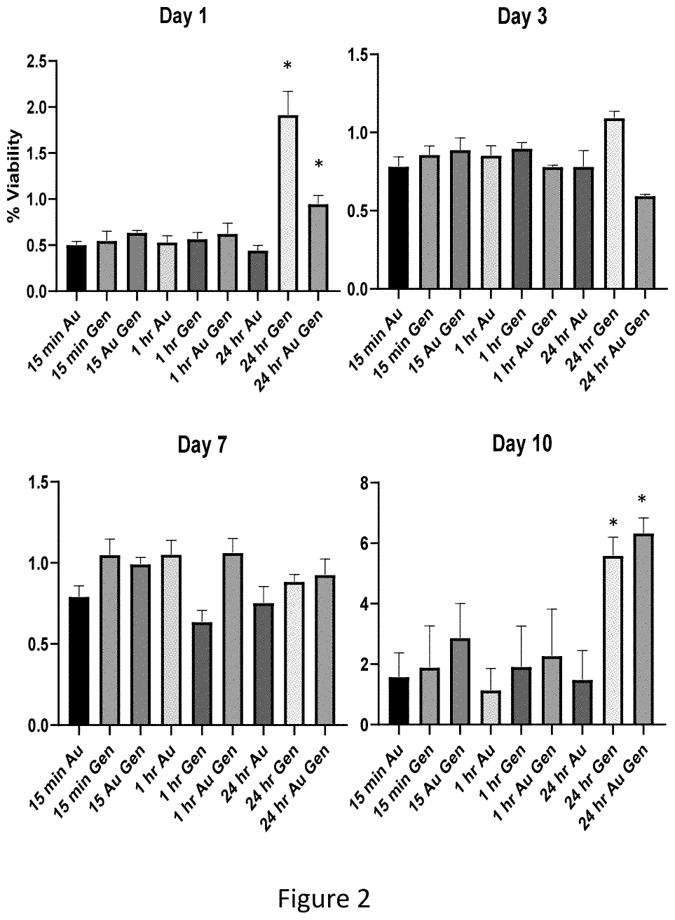

FIG. 2 shows the percent viability for each experimental group relative to decellularized untreated scaffolds (n=5). The scaffolds crosslinked with genipin for 24 hours, with or without gold nanoparticles, had significantly higher relative viability when compared to the uncrosslinked samples at day 1 and day 10. All other samples were not significantly higher than the uncrosslinked scaffolds.

### Example 9: dsDNA Assay

A total of 5 scaffolds (N=5) from 5 treatment types (untreated, 15 minutes gold and genipin, 1 hour gold and genipin, 24 hours gold and genipin, and 24 hours genipin only) were seeded with L929 mouse connective tissue fibroblasts and incubated for 1, 3, 7 and 10 days. Following cell culture, the discs were removed from their wells, gently rinsed, and frozen at 70° C. Samples were then lyophilized and submerged in papain digest and incubated at 60° C. for 24 hours. A Quant-iT PicoGreen double stranded DNA quantification assay (Thermo Fisher Scientific, Waltham, Mass., USA) was used to determine the cellularity of the scaffold. 25 μL of each papain digested sample were added to a 48-well plate. 225 μL of TE buffer (10 mM Tris-HCl, 1 mM EDTA, pH 7.5) and 250 μL of 2 μg/mL of PicoGreen reagent was added to each well and the plate was incubated for 5 minutes. Sample fluorescence was read at 480 nm excitation/520 nm using a Tecan Safire II plate reader. A Lambda DNA standard curve was used to determine DNA concentrations for the experimental samples.

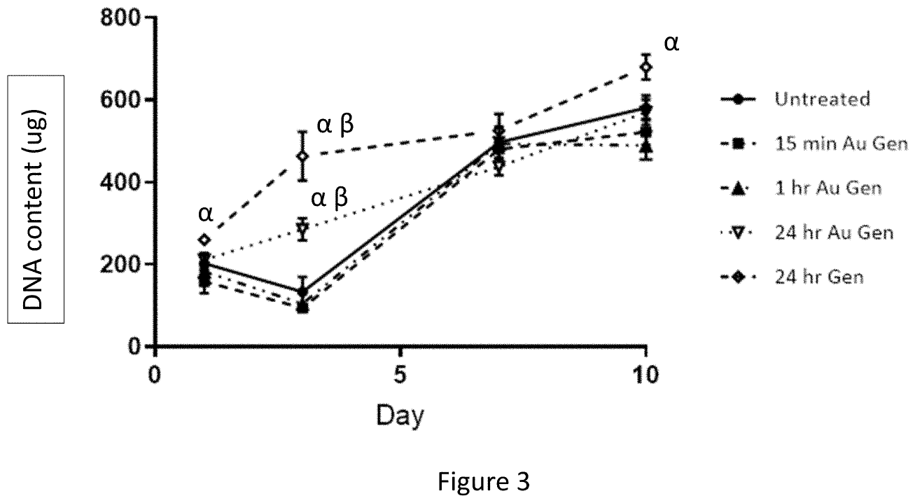

The PicoGreen assay used to determine dsDNA content of the cells attached to the tissue scaffold and is shown in FIG. 3. The sample with the 24 hour genipin crosslinking with no gold nanoparticles had the highest levels of dsDNA for all time points. It was significantly higher than all other samples except the 24 hour genipin with gold at day 3 and significantly higher than the samples crosslinked for 15 minutes or 1 hour at day 1 and day 10.

### Example 10: Scanning Electron Microscopy

SEM was utilized to visualize the attachment of the fibroblasts on the scaffold and observe the overall microstructure. The scaffolds were either uncrosslinked or crosslinked with 3 mM genipin with or without the presence of 20 nm AuNP. Scaffolds were plated with fibroblasts for 1 or 3 days and were then prepared by fixation in 0.1 M cacodylate buffer containing 2% glutaraldehyde and 2% paraformaldehyde. Samples were critically point dried and examined using a FEI Quanta 600 F Environmental SEM.

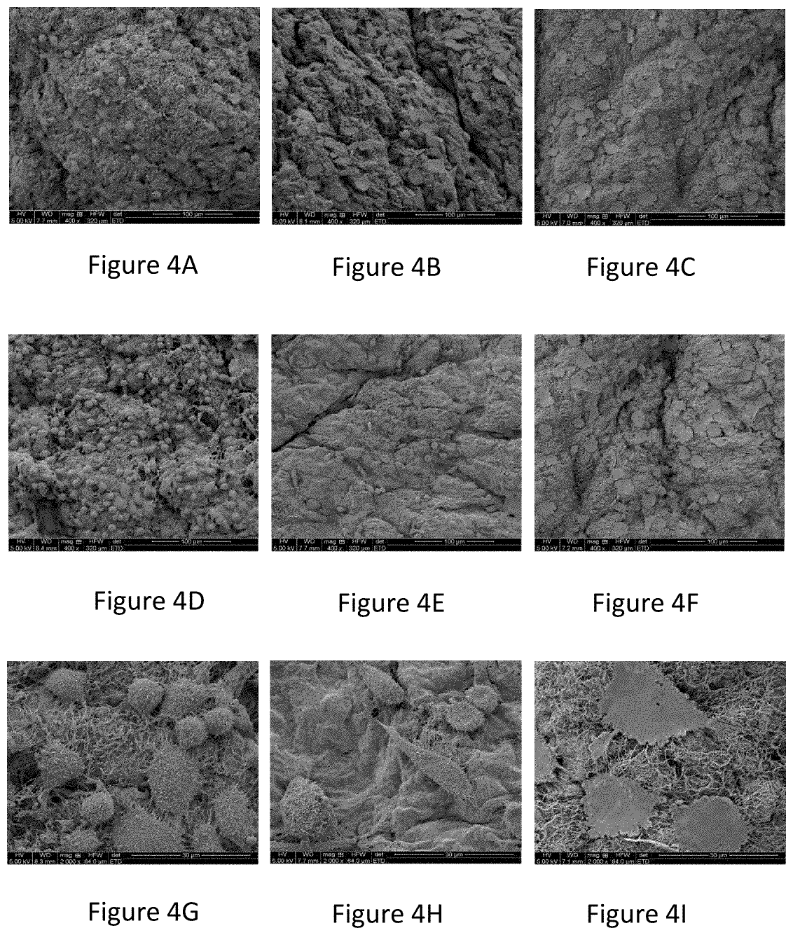

The SEM images demonstrate the presence of fibroblast cells on the scaffold. The images also show the cells plated on the scaffolds with genipin alone are flusher, and more tightly adhered to the scaffold in comparison to the more spherical cells on the uncrosslinked scaffold at both day 1 and day 3. This is especially visible at the day 3 timepoint and can be seen on images FIG. 4D and FIG. 4G versus FIG. 4E and FIG. 4I. The fibroblasts on the genipin and AuNP scaffolds are moderately leveled but remained more spherical than the fibroblasts attached to scaffold with genipin alone as seen in images FIG. 4B, FIG. 4F and FIG. 4H.

### Example 11: Additional Data

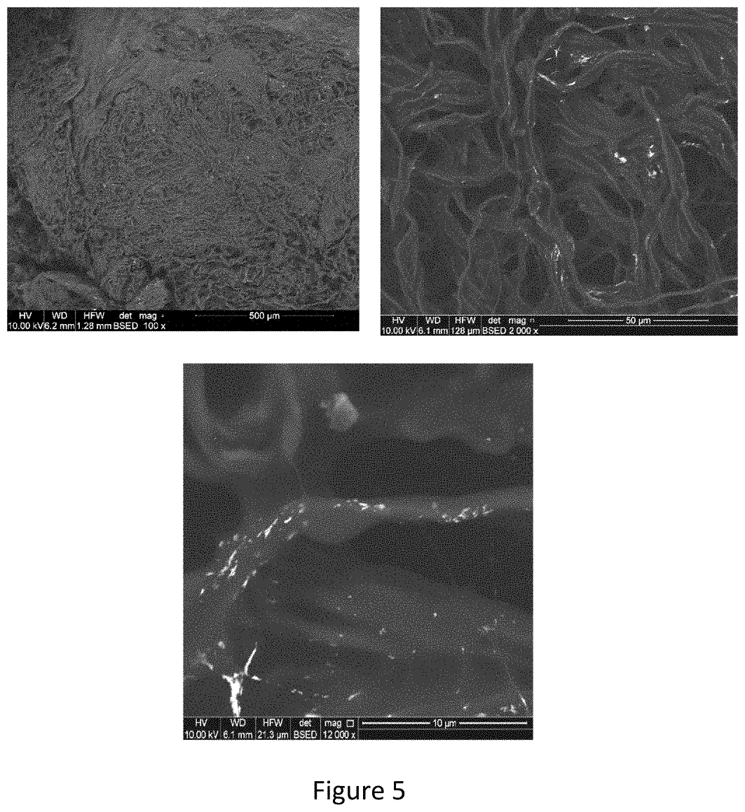

FIG. 5 is scanning electron microscopy images of gold nanoparticles conjugated with genipin to decellularized diaphragm tendon with crosslinking for 15 minutes.

### Example 12: Mechanistic Insights

The results of the disclosure demonstrated the ability of genipin to conjugate gold nanoparticles to a decellularized porcine tissue scaffold while also maintaining good biocompatibility. The gold nanoparticles, functionalized with amino groups, allowed the genipin to covalently cross-link between the amino residues on the nanoparticles and the amino groups on the tissue. The modified cyclic form of genipin resides stably within the extracellular collagen matrix adding bridges from adjacent fibers to the functionalized AuNPs.

The NAA results demonstrated a correlation between the crosslinking time and the amount of AuNPs attached to the scaffold. An interesting result is noted in that there is an increase in the amount of AuNPs from 15 minute to the 4 hour immersion times. However, there is no significant increase from the 4 hour to 8 hour time point followed by a significant increase at 24 hours. These biphasic results can be explained via the mechanism of genipin crosslinking. Genipin crosslinking occurs via two separate reactions involving different sites on the genipin molecule. The first reaction is a nucleophilic attack of the genipin C3 carbon atom from a primary amine group which occurs almost immediately. The second slower reaction is the nucleophilic substitution of genipin's ester group to form a secondary amide. The results clearly demonstrated genipin's biphasic reaction. In addition, the results also demonstrated that the functionalized AuNPs will bind to the tissue without the use of a crosslinker; however, the amount is significantly lower.

The DSC results provided additional confirmation on the binding ability of genipin. As shown in FIG. 1, genipin demonstrated higher denaturation temperatures with the increased crosslinking times (15 minutes vs. 24 hours) and with the higher genipin concentration (3 mM vs. 10 mM). The results also demonstrated the “two stage” binding ability of genipin in which the 4 hour and 8 hour crosslinking times displayed very similar denaturation temperatures. Genipin has previously been utilized to crosslink a collagen chitosan scaffold. The results showed that the longer crosslinking times and higher genipin concentrations led to increased mechanical strength in that scaffold. However, it was also seen that when genipin concentrations reached above 1% (w/v) the mechanical strength decreased.

The biocompatibility results of the present disclosure demonstrated that genipin is not cytotoxic as confirmed by previous results. On the contrary, the scaffolds with longer incubation times showed both an increase in cell numbers (FIG. 3) and cell proliferation (FIG. 2) when compared to untreated samples. This result was seen with or without the addition of the AuNPs meaning the genipin alone is associated with this increased cell growth. The enhanced cellular viability was demonstrated almost immediately with both biocompatibility assays showing a difference between the controls and the genipin treated scaffolds as early as 24 hours after plating the cells, suggesting the enhanced cellular capabilities are the results of improved cell adhesion. To confirm this, SEM images were acquired at day 1 and day 3 after the scaffolds were plated with fibroblast cells. The cells plated on the scaffolds crosslinked with genipin alone were flusher to the scaffold than the other two treatment types. The treatment of genipin allowed the cells to more quickly adhere and regain their spindle like appearance in comparison to the scaffolds left uncrosslinked or the scaffolds treated with genipin and gold.

Cell adhesion is a dynamic process involving interactions between cell cytoskeleton, extracellular matrix proteins, and peripheral membrane proteins. These adhesion protein complexes are crucial for the assembly of individual cells into the three-dimensional tissues and play an important role in further cell proliferation, viability, and differentiation. It is well documented that physical surface properties, including stiffness, can significantly influence cell attachment. Forces generated by the cytoskeleton are applied to membrane attachment sites. This can deform materials that lack a degree of stiffness but cannot move an attachment site on a rigid surface. Consequently, cell morphology and functions hinge on substrate stiffness. It was previously found that the use of genipin crosslinking increased surface roughness and stiffness on a hydrogel surface. This in turn resulted in better cell attachment, and better cell adhesion was associated with higher cell viability and proliferation. As shown in FIG. 2, there was an abrupt increase in 10 days 24 hour Gen and 24 hour Au Gen samples. In correlation, the DSC results showed the samples immersed in the genipin solution for 24 hours had a higher degree of crosslinking. This crosslinking most likely resulted in increased scaffold stiffness, and this may have contributed to the increased cell growth at 24 hours as shown in FIG. 2.

Without being limited by theory, there is evidence that the improved cell attachment may be the result of direct genipin interactions with the cells. The switch from spherical to flattened shapes was not only demonstrated on the fibroblasts growing on the scaffolds, but this phenomenon was also witnessed in the cells attached to the well plate directly adjacent to the scaffolds crosslinked with genipin for 24 hours. This is most likely the result of leaching of genipin, or other products of the crosslinking reaction, from the scaffold to the nearby cells. The exact mechanism for this is unclear, and there were no other cases of this phenomenon cited in the literature. On the contrary, others have previously hypothesized that genipin may impair cell adhesion as it halved the mRNA expression of essential cell adhesion protein integrin β1 in chondrocytes (Wang, et al., J. Biomed. Mater. Res. Part B Appl. Biomater. 2011, 97, 58-65.). However, the results of the present disclosure clearly demonstrate genipin supporting fibroblasts as they attach, spread out, and flatten both on the scaffold and neighboring to it.

While the invention has been described in connection with specific embodiments thereof, it will be understood that the inventive methodology is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth and as follows in scope of the appended claims.

When introducing elements of the present invention or the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description shall be interpreted as illustrative and not in a limiting sense.

1. A treatment composition comprising a nanoparticle composition comprising nanoparticles functionalized with surface amine groups and a crosslinking composition comprising genipin.

2. The treatment composition of claim 1, wherein the composition has a genipin concentration from about 0.01 mM to about 10 mM.

3-6. (canceled)

7. The treatment composition of claim 1, wherein the composition further comprises a buffer, an antimicrobial agent, an anti-inflammatory agent, a cell culture media, or a combination thereof.

8-9. (canceled)

10. The treatment composition of claim 1, wherein the nanoparticles have a mean diameter of from about 5 nm to about 100 nm.

11-12. (canceled)

13. The treatment composition of claim 1, wherein the nanoparticles are metallic nanoparticles, ceramic nanoparticles, or polymer nanoparticles.

14. (canceled)

15. The treatment composition of claim 13, wherein the metallic nanoparticles are gold nanoparticles.

16-23. (canceled)

24. The treatment composition of claim 1, wherein the nanoparticles are functionalized with surface amino groups via addition of 2-mercaptoethylamine.

25. (canceled)

26. A kit for crosslinking nanoparticles to a tissue graft, the kit comprising a nanoparticle composition comprising nanoparticles, a functionalization component comprising a ligand having a thiol group and an amine group, a crosslinking composition comprising genipin, a buffer solution, and instructions for using the kit to crosslink the nanoparticles to the tissue graft, wherein the instructions comprise instructions to contact the nanoparticle composition with the functionalization component to form a functionalized nanoparticle composition, and combine the crosslinking composition, the functionalized nanoparticle composition, and the buffer solution to form a incubating composition and contact the tissue graft with the incubating composition.

27. The kit of claim 26, wherein the crosslinking composition comprising genipin is provided as a dry powder.

28. (canceled)

29. The kit of claim 26, wherein the kit further comprises an antimicrobial agent, an anti-inflammatory agent, a cell culture media, or a combination thereof.

30-31. (canceled)

32. The kit of claim 26, wherein the nanoparticles have a mean diameter of from about 5 nm to about 100 nm.

33-34. (canceled)

35. The kit of claim 26, wherein the nanoparticles are metallic nanoparticles, ceramic nanoparticles, polymer nanoparticles, or a combination thereof.

36. (canceled)

37. The kit of claim 35, wherein the metallic nanoparticles are gold nanoparticles.

38-44. (canceled)

45. The kit of claim 26, wherein the nanoparticles are functionalized with surface amino groups via addition of 2-mercaptoethylamine.

46. A method of crosslinking nanoparticles to a tissue graft, the method comprising:
a) preparing a treatment composition of claim 1 optionally further comprising a buffer solution;
b) contacting the tissue graft with the treatment composition for at least 15 minutes; and
c) rinsing the tissue graft.

47-48. (canceled)

49. The method of claim 46, wherein the incubation is about 15 minutes.

50. The method of claim 46, wherein the tissue graft is a soft tissue.

51-52. (canceled)

53. The method of claim 46, wherein the tissue graft comprises urinary bladder, small intestine, dermis, mesothelium, heart valve, or pericardium tissue.

54-56. (canceled)

57. The method of claim 46, wherein the nanoparticles crosslinked to the tissue graft have a concentration of 50 μg/g to 400 μg/g.

58. (canceled)

59. The method of claim 46, wherein the method is implemented within a surgical suite.

60-68. (canceled)
